(19) United States
(12) Patent Application Publication
(10) Pub. No.: US 2023/0317269 A1
(43) Pub. Date: Oct. 5, 2023

(54) System and Method For Evaluating, Monitoring, Assessing and Predicting Ambient and Health Conditions

(71) Applicants: Steven Isaac Davis, Berea, KY (US); Nichol Marie Case, Berea, KY (US)

(72) Inventors: Steven Isaac Davis, Berea, KY (US); Nichol Marie Case, Berea, KY (US)

(21) Appl. No.: 18/128,563

(22) Filed: Mar. 30, 2023

Related U.S. Application Data
(60) Provisional application No. 63/326,726, filed on Apr. 1, 2022.

(51) Int. Cl. G16H 40/63; G06N 5/022; G06Q 30/0601

(52) U.S. Cl. CPC G16H 40/63; G06N 5/022; G06Q 30/0633

(57) ABSTRACT

System and method for evaluation and monitoring of environment and health of an occupant is provided. The system comprises a first sensor group that collects environment data, a second sensor group that collects health and wellness data, and a medical files dataset comprising at least one medical file, relating to the occupant. The system comprises an ambient health data platform configured to generate a monitoring dataset based on the first sensor group, the second sensor group, and the medical files dataset; create an occupant health and wellness evaluation based on the monitoring dataset; perform a predictive analysis to recommend enhancements to the monitoring dataset to improve the health and wellness evaluation of the occupant; and provide integrated e-commerce capabilities for the occupant to order, fulfill, and integrate the recommended enhancements. The monitoring dataset is improved based on incorporation of the recommended enhancements by the occupant.

### CROSS-REFERENCE TO RELATED APPLICATION

This application claims the benefit of U.S. Provisional Application No. 63/326,726 filed Apr. 1, 2022.

### TECHNOLOGICAL FIELD

The present disclosure relates generally to environmental monitoring and health and wellness monitoring, and more particularly, the present disclosure relates to systems and methods for evaluation and monitoring of the combined environmental data and health data of an occupant to manage and predict occupant needs.

### BACKGROUND

Managing healthcare has become very challenging for several reasons. The rapid advances in medical technologies and the proliferation of choices with respect to providers and modalities of treatment can be confusing to an average patient, and even more so to elderly patients or patients whose ability to comprehend these choices has been impaired. In general, the healthcare system is structured to handle issues or problems after they have occurred rather than analyzing the patient data to prevent and predict health outcomes. Further, the healthcare system does not have the capacity or collection techniques to consider ambient, environmental, and social factors that impact health of a user. Moreover, when data is collected from health monitoring devices, the data gathered from different sensors is isolated from other collected or available data and not integrated into a more complete monitoring and predictive system.

The proliferation of home health monitoring devices and “smart home” devices has exasperated this problem. For example, such health monitoring devices and smart home devices selected by users are designed to only interact with corresponding manufacturer applications or other devices supported by manufacturer because the device manufacturers have a profit motive for driving further purchases within their product lines. Further, an occupant or a user centric viewpoint of the value of shared data has not permeated any industry. In particular, the home health, smart home, personal sensor, safety monitoring, wellness and other industries have not recognized that safety, health, and welfare of a user or an occupant is improved with the holistic evaluation of their home and themselves.

The use of wearable and sensing devices to observe or monitor physical activities, whether for health, sports monitoring, or medical rehabilitation, has surged due to the advancement in sensing technology, more affordable integrated circuits and the growth of connectivity technologies. The wearable and sensing devices may be deployed in biomedical applications to provide wellness and health benefits by utilizing data processing techniques.

For example, the wearable devices may be used to monitor health conditions of a user by monitoring parameters, such as temperature, positioning, and electrical bio-signals as electrocardiograms (ECGs), electromyograms (EMGs), and electroencephalograms (EEGs). In this manner, the wearable devices may track daily routine, sleep cycle, pulse and blood pressure information, and other activities performed by the user. However, generating a health report for diagnosis, prevention, examination, or cure of any disease, based merely on sensor data acquired by wearable devices, may be highly inaccurate. In particular, analyzing health condition of the user based on the wearable device may be prone to incorrect medical analysis owing to lack of history information and information relating to other changes in physical or health condition of the user. In addition, the wearable devices may also fail to predict any potential future occurrence of diseases or future health condition for the user.

In recent days, a user may utilize a plurality of wearable devices and other sensing devices to collect or track different information, for example, heart rate (HR), HR variability, respiratory rate, number of steps, distance traveled, pace, maximal oxygen consumption, calories burned, calorie intake, sleep stages (REM, light and deep), sleep quality, food and liquid intake, running speed, elevation, climbing rate, biking rate, swimming rate, and so forth. However, data produced by different sensing devices and the wearable devices are isolated from other collected or available data. As a result, the user may be unaware of their holistic health condition.

Typically, each individual wearable device and sensing device may be designed to only interact with corresponding manufacturer's applications and/or an application providing profit motive for the manufacture. Further, data collected by individual wearable device and sensing devices may not provide a holistic view or a manner of change in a particular parameter sensed by a sensing device over days, months or years. Moreover, such data may not be in user-readable format, thereby increasing user frustration and rendering the use of such wearable and sensing devices futile. To this end, a user-centric data may not be provided to the user.

Further, the wearable and sensing devices fail to evaluate ambient conditions or characteristics, such as living conditions, assisted living equipment, and so forth, which may impact or improve health of the user and generating a health report for the user.

It would therefore be desirable to have a system that provides a means to address some of the aforementioned problems. Most current systems provide only current health status of the user, without consideration of the user's environment. There is not currently a system or process that evaluates the occupant, the dwelling and the social characteristics that impact or can improve health, welfare, and safety for those who may have reduced capabilities due to age, infirmity, disease, injury, mental status or other limitations.

Therefore, there is a need to overcome the limitations associated with conventional health monitoring systems.

### BRIEF SUMMARY OF SOME EXAMPLE EMBODIMENTS

In order to solve foregoing problem, the present disclosure may provide a system, a method and a computer programmable product for progressive evaluation and monitoring of environment and health of an occupant. The current invention provides for learned and predictive recommendations to users or caregivers of the users for preventive health and risk avoidance based upon past, present, and anticipated future user data.

Some embodiments are based on the understanding that conventional sensing devices and wearable devices provide only current health status of a user (referred to as an occupant, hereinafter), without consideration of the user's environment, such as their home (referred to as dwelling, hereinafter), support community, medical personnel, and so forth.

Some embodiments are based on the understanding that safety, health and welfare of an occupant may be improved based on identification and evaluation of their ambient conditions and characteristics comprising data relating to, for example, home, social characteristics, and personal health evaluation. Subsequently, the present disclosure provides techniques for evaluating the occupant, a dwelling of the occupant, and the social characteristics for occupants that may have reduced capabilities due to age, infirmity, disease, injury, mental status, or other limitations.

It is an object of the present disclosure to provide an integrated and automated evaluation system that considers status quo (i.e., present health condition) for the occupant, condition of the dwelling of the occupant, characteristics of environment of the occupant, and other social and support circle that surrounds the occupant. To this end, the system may identify a risk, or a potential improvement to the status quo of the occupant. The system may also enable early detection and/or prevention of certain diseases and injuries based on the evaluation. It is an objective of the present disclosure to provide techniques for identifying ambient conditions and characteristics of the occupant that indicates health and wellness status of the occupant based on a combination of environment data and health and wellness data of the occupant and predicts future health condition possibilities to ensure overall well-being of the occupant.

The present disclosure integrates data obtained from various home monitoring sensors, health monitoring sensors, wearable devices, and other personally reported health data. The system may provide insights into health condition of the occupant for current monitoring and future predictive/preventative analytics for risk avoidance.

It is an object of the present disclosure to establish and retain data collected from various sensors and wearable devices associated with an occupant. Further, the data may be used for retrospective and predictive analysis of experience of home life of the occupant, and to evaluate health condition of the occupant periodically and recommend future improvements to the occupant. The present disclosure provides techniques for learned and predictive recommendations for the occupant for preventive health and risk avoidance based upon past, present, and anticipated future data.

It is an object of the present disclosure to provide a detailed recommendation and immediate ordering/scheduling benefits to the occupant in order to streamline identification, purchase, installation, and setup of recommendations. In this manner, the system may enable patients to manage their health issues more effectively, provide a way for family members and friends to support the health of the patient, and provide caregivers and family predictive recommendations for future risk avoidance, better health, and better living.

A system, a method and a computer programmable product are provided for a progressive evaluation and monitoring of environment and health of an occupant.

In one aspect, a system for progressive evaluation and monitoring of environment and health of an occupant is provided. The system comprises a first sensor group to collect environment data relating to the occupant, a second sensor group to collect health and wellness data relating to the occupant, a medical files dataset comprising at least one medical file relating to the occupant, and an ambient health data platform. The first sensor group comprises a plurality of dwelling sensors, and the second sensor group comprises a plurality of health sensors. The ambient health data platform is configured to generate a monitoring dataset based on the first sensor group, the second sensor group, and the medical files dataset. The ambient health data platform is configured to create an occupant health and wellness evaluation based on the monitoring dataset. The ambient health data platform is configured to perform a predictive analysis to recommend enhancements to the monitoring dataset to improve the health and wellness evaluation of the occupant. The ambient health data platform is configured to provide integrated e-commerce capabilities for the occupant to order, fulfill, and integrate the recommended enhancements. The monitoring dataset is improved based on incorporation of the recommended enhancements by the occupant.

In additional system embodiments, the plurality of dwelling sensors comprises at least a camera.

In additional system embodiments, the plurality of health sensors comprises at least one of: one or more occupant wearable sensors and one or more home health devices.

In additional system embodiments, the at least one medical file comprises at least one of: personal health records, electronic medical record, medical history, or third-party data obtained from an occupant caregiver, relating to the occupant.

In additional system embodiments, the third-party data obtained from the occupant caregiver includes data from at least one of: family members, or medical professionals.

In another aspect, a system for progressive evaluation and monitoring of environment and health of an occupant is provided. The system comprises a first sensor group to collect environment data relating to the occupant, a second sensor group to collect health and wellness data relating to the occupant, a medical files dataset, and an ambient health data platform. The first sensor group comprises a plurality of dwelling sensors, the second sensor group comprises a plurality of health sensors including occupant wearable sensors and home health devices, and the medical files dataset comprises at least one of: personal health records, electronic medical record, available medical history, and third-party data obtained from an occupant caregiver, relating to the occupant. The ambient health data platform is configured to generate a monitoring dataset based on the first sensor group, the second sensor group, and the medical files dataset. The ambient health data platform is configured to create an occupant health and wellness evaluation based on the monitoring dataset. The ambient health data platform is configured to perform a predictive analysis to recommend enhancements to the monitoring dataset to improve the health and wellness evaluation of the occupant. The ambient health data platform is configured to provide integrated e-commerce capabilities for the occupant to order, fulfill, and integrate the recommended enhancements. The monitoring dataset is improved based on incorporation of the recommended enhancements by the occupant.

In additional system embodiments, the plurality of dwelling sensors comprises at least a camera.

In additional system embodiments, the third-party data obtained from the occupant caregiver includes data from at least one of: family members, or medical professionals.

In yet another aspect, a method for progressive evaluation and monitoring of environment and health of an occupant is provided. The method may be performed using a system comprising a first sensor group to collect environment data relating to the occupant, a second sensor group to collect health and wellness data relating to the occupant, a medical files dataset, and an ambient health data platform. The first sensor group comprises a plurality of dwelling sensors, the second sensor group comprises a plurality of health sensors, and the medical files dataset comprise at least one medical file relating to the occupant. The method comprises generating, using the ambient health data platform, a monitoring dataset based on a first sensor group, a second sensor group, and a medical files dataset. The method comprises creating, using the ambient health data platform, an occupant health and wellness evaluation based on the monitoring dataset. The method comprises performing, using the ambient health data platform, a predictive analysis to recommend enhancements to the monitoring dataset to improve the health and wellness evaluation of the occupant. The method comprises providing, using the ambient health data platform, integrated e-commerce capabilities for the occupant to order, fulfill, and integrate the recommended enhancements. The method comprises improving, using the ambient health data platform, the monitoring dataset based on incorporation of the recommended enhancements.

In additional method embodiments, the occupant health and wellness evaluation is performed by an evaluator.

In additional method embodiments, the plurality of health sensors comprises at least one of: one or more occupant wearable sensors, or one or more home health devices.

In additional method embodiments, the at least one medical file comprises at least one of: personal health records, electronic medical record, medical history, or third-party data obtained from an occupant caregiver, relating to the occupant.

In additional method embodiments, the third-party data obtained from the occupant caregiver includes data from at least one of: family members, or medical professionals.

In additional method embodiments, the method further comprises performing, using the ambient health data platform, an occupant health and wellness re-evaluation based on the improved monitoring dataset after the incorporation of the recommended enhancements.

In additional method embodiments, the recommended enhancements comprise an additional health sensor for environment and health monitoring of the occupant.

In additional method embodiments, the monitoring dataset comprises historical data obtained from the plurality of dwelling sensors, the plurality of health sensors, and the medical files dataset.

In additional method embodiments, the ambient health data platform receives data from the first sensor group and the second sensor group via a communication network.

In additional method embodiments, the occupant health and wellness evaluation includes an assessment of physical capabilities of the occupant.

In additional method embodiments, the occupant health and wellness evaluation includes an assessment of the plurality of dwelling sensors, the plurality of health sensors, and the medical files dataset of the occupant.

In yet another aspect, a computer programmable product for progressive evaluation and monitoring of environment and health of an occupant is provided. The computer programmable product comprises a non-transitory computer readable medium having stored thereon computer executable instructions, which when executed by one or more processors, cause the one or more processors to carry out operations. The operations comprise generating, using the ambient health data platform, a monitoring dataset based on a first sensor group, a second sensor group, and a medical files dataset. The first sensor group collects environment data relating to the occupant, the second sensor group collects health and wellness data relating to the occupant, and the medical files dataset comprise at least one medical file relating to the occupant. The first sensor group comprises a plurality of dwelling sensors, and the second sensor group comprises a plurality of health sensors. The operations comprise creating, using the ambient health data platform, an occupant health and wellness evaluation based on the monitoring dataset. The operations comprise performing, using the ambient health data platform, a predictive analysis to recommend enhancements to the monitoring dataset to improve the health and wellness evaluation of the occupant. The operations comprise providing, using the ambient health data platform, integrated e-commerce capabilities for the occupant to order, fulfill, and integrate the recommended enhancements. The operations comprise improving, using the ambient health data platform, the monitoring dataset based on incorporation of the recommended enhancements.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

Having thus described example embodiments of the invention in general terms, reference will now be made to the accompanying drawings, which are not necessarily drawn to scale, and wherein:

FIG. 1 illustrates a network environment in which a system for progressive evaluation and monitoring of environment and health of an occupant is implemented, in accordance with an embodiment of the present disclosure;

FIG. 2 illustrates a block diagram of the system for progressive evaluation and monitoring of environment and health of the occupant, in accordance with an embodiment of the present disclosure;

FIG. 3 illustrates a support network associated with an occupant and an ambient health data platform, in accordance with an embodiment of the present disclosure;

FIG. 4 illustrates a flow chart of a method for evaluation and assessing of environment of a dwelling and health of an occupant, in accordance with an embodiment of the present disclosure;

FIG. 5 illustrates a flow diagram of a method for monitoring a dwelling and health and wellness status of an occupant, in accordance with an embodiment of the present disclosure; and

FIG. 6 illustrates a method for progressive evaluation and monitoring of environment and health of an occupant, in accordance with an embodiment of the present disclosure.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present disclosure. Thus, use of any such terms should not be taken to limit the spirit and scope of embodiments of the present disclosure.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

### Definitions

The term “occupant” may refer to a person or an entity that may use the provided system for progressive evaluation and monitoring of environment and health of an occupant. In an example, the occupant may be a person having a health condition, i.e., a patient having a diseases that may use the system for better managing of the disease and prevent occurrence of future diseases. In another example, the occupant may be a healthy person that may use the system for improving health, identify diseases early and prevent occurrence of future diseases.

The term “dwelling” may refer to a physical living space of an occupant. Examples of the dwelling may include, but are not limited to, a house, a flat, a hospital, an assisted living center, a care shelter, or other place of residence.

The term “health evaluation” may refer to a detection of a health related risk to the occupant.

The term “first sensor group” may refer to a plurality of a first type of sensors. For example, the first sensor group includes a plurality of dwelling sensors. In particular, the plurality of dwelling sensors are positioned or deployed within a dwelling of an occupant. The plurality of dwelling sensors of the first sensor group are configured to collect environment data relating to the occupant, particularly, environment data within dwelling of the occupant.

For example, one or more sensors from the plurality of dwelling sensors may be associated with a security or surveillance device, for example, smoke sensors, temperature sensors and carbon oxide (CO) sensors may be associated with a fire detection device; and face recognition sensor, motion detection, and camera may be associated with a security surveillance device. Moreover, one or more sensors from the plurality of dwelling sensors may be associated with an appliance, for example, smoke sensors, imaging devices, timers, and temperature sensors may be associated with an oven; and imaging devices, pressure sensors, touch sensors and temperature sensors may be associated with a fridge. Examples of the plurality of dwelling sensors may include, but are not limited to, camera, refrigerator sensors, oven sensors, smoke or fire or CO detection sensors, leak or moisture detection sensors, window and door open or close detection sensors, motion sensors, garage door sensors, temperature sensors, light sensors, water usage detection sensors, humidity sensors, video doorbell, weather sensor, gas leakage sensor, other equipment sensors and emergency notification or siren sensors.

The term “second sensor group” may refer to a plurality of second type of sensors. For example, the second sensor group includes a plurality of health sensors. In particular, the plurality of health sensors are positioned or deployed on the occupant or may track or monitor parameters of the occupant. The plurality of health sensors of the second sensor group are configured to collect health and wellness data relating to the occupant, particularly, physical parameters indicative of health and wellness of the occupant.

For example, one or more sensors from the plurality of health sensors may be associated with one or more occupant wearable sensors or a wearable device. Examples of the occupant wearable sensors may include, but are not limited to, smart watch, headphones, finger ring, health band, fitness band, smart patch, biosensor, body area network device, smart phone, smart jewelry, AR/VR headsets, hearing aids, web-enabled glasses, smart clothing and implantables. Moreover, sensors associated with occupant wearable sensors may include, for example, accelerometer, gyroscope, magnetometer, barometric pressure sensor, ambient temperature sensor, heart rate monitor, oximetry sensor, skin conductance sensor, skin temperature sensor, global positioning sensor (GPS), optical sensor, sleep cycle and activity sensors, and stress sensors. Moreover, one or more sensors from the plurality of health sensors may be associated with one or more home health devices including, for example, electrocardiography (ECG) or electrokardiographie (EKG) device, ventilators, blood pressure cuff, glucometer, weighing machines, stethoscope, oxygen concentrators, anesthesia delivery machines, dialysis machines, infusion pump machines, lung capacity detection devices, respiration sensors, and other medical and health care devices.

The term “medical files dataset” may refer to a collection of data that pertains to a specific medical condition, treatment, or procedure, indicating a medical record of an occupant. The medical files dataset may include various types of information, such as patient demographic data, medical histories, past medical reports, prescriptions, injuries, diseases, clinical trial results, laboratory test results, imaging data, treatments and/or procedures performed, and other types of medical information.

For example, medical files for the occupant may be generated based on clinical trials, observational studies, or other types of medical research performed on the occupant, or other treatment or procedures performed on the occupant. In an example, the medical files dataset of the occupant may be obtained from various sources, including hospitals, clinics, research centers, and other healthcare organizations.

### End of Definitions

A system, a method and a computer program product are provided for progressive evaluation and monitoring of environment and health of an occupant.

In certain cases, a user or an occupant may use different health monitoring devices to monitor different physical parameters or different diseases. To this end, such different health monitoring devices are isolated from each other and fail to provide holistic support for accurate diagnosis, prevention, and cure for occupants. For example, the different health monitoring devices may require different memberships to provide evaluation data regarding certain physical parameters. As a result, the occupant may have to buy several memberships in order to monitor and evaluate different physical parameters and use them for any diagnosis. It may be highly inconvenient to manage different health monitoring devices and may also cause the occupant to expend more money.

Further, the conventional health monitoring devices fail to consider ambient conditions and characteristics of the occupant that is based on a combination of environment conditions and health, wellness and social characteristic of a user or an occupant in determining or predicting a health condition or a disease. For example, poor environment or daily routine might contribute to diseases and may impact on ability to stay healthy. Conventional health monitoring devices merely identify current symptoms of the occupant to identify an anomaly and identify possible diseases or health risk. Therefore, the conventional health monitoring devices fail to identify early indicators of a disease and only notify the occupant when a symptom of a disease may be detected or disease may have occurred. Such delayed warning of the disease may not be helpful. Moreover, data collected by the conventional health monitoring devices may also not help in diagnosis of a disease accurately.

The system, method, and computer-programmable product described in the present disclosure enable progressive evaluation and monitoring of environment and health of an occupant. The system may be configured to integrate sensor data from various sensors deployed in dwelling or environment of the occupant and health sensors associated with occupant to identify health condition of the occupant accurately. Further, the system may be configured to recommend enhancements to improve health and/or environment of the occupant. In addition, the system may enable the occupant to fulfill the required enhancements to improve their health. The system may be configured to periodically monitor health of the occupant based on incorporation of the enhancements and other changes in the parameters of the occupant and/or dwelling.

In this manner, the system may be configured to generate monitoring data by collecting information from every relevant data source, such as plurality of dwelling sensors, occupant, caregiver, historical medical records, plurality of health sensors, home devices, personal wearable devices, and other sources. Further, based on data processing techniques, occupant monitoring data may be evaluated, and some predictive enhancements may be provided to the occupant for betterment of health conditions and/or quality of living. In certain cases, the integrated monitoring data may be used for medical diagnosis, for example, by a medical professional. Using the monitoring data, the medial diagnosis may be accurate as the monitoring data indicates ambient conditions and characteristics of the occupant comprising several social as well as biological factors of the occupant, thereby enabling deeper understanding of well-being of the occupant.

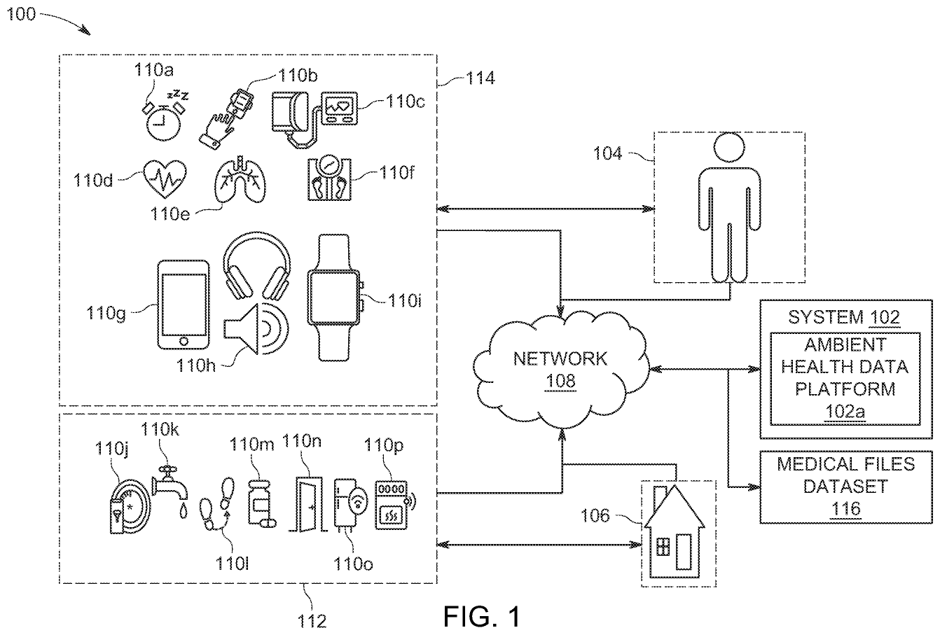

FIG. 1 illustrates a network environment 100 in which a system 102 for progressive evaluation and monitoring of environment and health of an occupant 104 is implemented, in accordance with an example embodiment. The network environment 100 comprises the system 102, the occupant 104, a dwelling 106, and a communication network 108. Further, the network environment 100 comprises a plurality of sensors, depicted as sensors 110a, 110b, 110c, 110d, 110e, 110f, 110g, 110h, 110i, 110j, and 110k (collectively referred to as sensors 110, hereinafter). For example, the plurality of sensors 110 may be a part of the system 102 and/or coupled to entities of the system 102.

In some example embodiments, the system 102 may include processing means such as a central processing unit (CPU), storage means such as on-board read only memory (ROM) and random access memory (RAM), and coupled sensors, for example, the sensors 110.

In an embodiment, the system 102 may include the sensors 110, for example, as a part of a comprehensive monitoring and evaluation system, a monitoring and evaluation app in a mobile device, and the like. In this regard, the system 102 may be communicatively coupled to the components shown in FIG. 1 to carry out the desired operations and wherever required modifications may be possible within the scope of the present disclosure.

The dwelling 106 may be a house, an apartment or any other place of residence and/or work that is used to provide temporary and/or permanent shelter to the occupant 104. In an embodiment, the dwelling 106 provides accommodation to the occupant 104. In addition, the occupant 104 may be any person, such as a healthy person, a patient, or any person who needs health and wellness monitoring, or assistance and support for day-to-day activities.

In an example, the system 102 may include the sensors 110. In another example, the system may be connected with the sensors 110, via the communication network 108. For example, the system 102 may include a first sensor group 112 that collects environment data relating to the occupant 104. In particular, the first sensor group 112 comprises a plurality of dwelling sensors (such as, weight sensor 110j, water usage or leakage sensor 110k, steps counter 1101, medicine consumption sensors 110m, door sensors 110n, smart fridge 110o, and smart oven 110p). The dwelling sensors that may be positioned in the dwelling 106 of the occupant. The environment data collected by the dwelling sensors may indicate a state of an environment or state within the dwelling 106 in which the occupant 104 resides. Further, the sensors 110 may include a second sensor group 114 that collects health and wellness data relating to the occupant 104. In particular, the second sensor group 114 comprises a plurality of health sensors that may be associated with, connected to, or worn by the occupant 104. The health and wellness data collected by the health sensors may include readings pertaining to current body or physical parameters relating to the occupant 104, thereby indicating current health condition of the occupant 104.

Further, the network environment 100 comprises a medical files dataset 116. In an example, the system 102 may be connected to the medical files dataset 116, via the communication network 108. In another example, the system 102 may comprise the medical files dataset 116. The medical files dataset 116 may include at least one medical file relating to the occupant 104. For example, the medical file may include historical medical records, such as prescriptions, treatments, clinical trial results, laboratory reports, etc. relating to the occupant 104. For example, the system 102 may acquire or obtain medical files dataset 116 from a medical database, such as associated with a hospital, a research lab, a pathology lab, and a clinic.

The communication network 108 may be wired, wireless, or any combination of wired and wireless communication networks, such as cellular, Wi-Fi, internet, local area networks, or the like. In some embodiments, the communication network 108 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks (for e.g. LTE-Advanced Pro), 5G New Radio networks, ITU-IMT 2020 networks, code division multiple access (CDMA), wideband code division multiple access (WCDMA), wireless fidelity (Wi-Fi), wireless LAN (WLAN), Bluetooth, Internet Protocol (IP) data casting, satellite, mobile ad-hoc network (MANET), and the like, or any combination thereof.

In operation, the system 102 includes an ambient health data platform 102a configured to perform various operations associated with the system 102. In an example, the ambient health data platform 102a may be configured to generate a monitoring dataset based on the first sensor group 112, the second sensor group 114, and the medical files dataset 116. The ambient health data platform 102a receives environment data relating to the dwelling 106 of the occupant 104 through a plurality of dwelling sensors from the first sensor group 112, based on one or more network technologies. Moreover, the ambient health data platform 102a receives health and wellness data relating to the occupant 104 through a plurality of health sensors from the second sensor group 114, based on one or more network technologies. Further, the ambient health data platform 102a extracts at least one medical file relating to the occupant 104 from the medical files dataset 116. For example, the medical file may store past, planned and/or ongoing medical record, medical history, past medical reports, prescriptions, laboratory test reports, past injuries, past diseases, emergencies caused in past, and the like.

For example, the one or more network technologies, such as the network 108, may include Bluetooth, Wi-Fi, Zigbee, Z-Wave, proprietary mesh network, proprietary data API and the like.

For example, the ambient health data platform 102a may be configured to generate the monitoring dataset based on the environment data received from the first sensor group 112, the health and wellness data received from the second sensor group 114 and the medical file(s) received from the medical files dataset 116. For example, the monitoring dataset may include available health record of the occupant 104 and condition or change in condition of the dwelling 106. For example, the monitoring data may include information relating to the occupant 104 and corresponding dwelling 106 to define environment and health conditions that are wholly specific and personalized to the data received relating to the occupant 104 and the dwelling 106. The ambient health data platform 102a may utilize the medical file, the environment data and the health and wellness data to generate monitoring dataset, and identify an initial baseline condition of health, wellness, environment and surroundings of the occupant 104, such as diseases and/or social factors affecting health and well-being of the occupant 104.

Further, the ambient health data platform 102a may be configured to create an occupant health and wellness evaluation based on the monitoring dataset. In an example, the ambient health data platform 102a may perform evaluation of the health condition of the occupant 104 and/or condition of the dwelling 106 based on the health and wellness data, the environment data and the medical file(s) to establish baseline status or current condition of the occupant 104 and/or the dwelling 106 in the ambient health data platform 102a. in an example, the ambient health data platform 102a may perform evaluation of the occupant 104, the dwelling 106 and the medical files using one or more artificial intelligence-based algorithms. The occupant health and wellness evaluation may refer to information indicating the present capabilities of the occupant 104 and current condition of dwelling 106. For example, based on evaluation of health and wellness data of the occupant 104 and identifying high blood pressure readings, an evaluation of heart problems may be made for the occupant 104; or based on evaluation of environment data and identifying water leakage readings, an evaluation of presence of moisture in the dwelling 106 may be made.

The ambient health data platform 102a may be configured to perform a predictive analysis to recommend enhancements to the monitoring dataset to improve the health and wellness evaluation of the occupant 104. The ambient health data platform 102a may perform predictive analysis of the health conditions of the occupant 104, capabilities and limitations of the occupant 104, and current state of the dwelling 106, to recommend modifications and enhancements for augmentation in the dwelling 106 or to be associated with the occupant. In an example, the predictive analysis may be used to predict any future risk for the occupant 104 from current evaluated monitoring dataset, i.e., current health condition of the occupant 104 and current condition of the dwelling 106.

The ambient health data platform 102a may be configured to provide integrated e-commerce capabilities for the occupant 104 to order, fulfill, and integrate the recommended enhancements. For example, the monitoring dataset is improved based on incorporation of the recommended enhancements by the occupant. In an example, based on the predictive analysis of the monitoring dataset or the occupant health and wellness evaluation, the ambient health data platform 102a may generate a recommendation, such as installing more accurate or advanced sensors for example, install blood pressure monitoring cuff by replacing optical based blood pressure monitoring device for more accurate and close monitoring of blood pressure. In such a case, the ambient health data platform 102a may provide provisions for direct purchase and provisioning of the additional sensors, such as the blood pressure cuff, to be installed in the dwelling 106.

For example, if the occupant 104 has a limiting condition (such as “reduced mobility”) and their dwelling 106 currently has stairs that must be used to enter the front door, but the garage entry has only 1 step to enter the house, the recommendations may include both “install ramp to front door” and “avoid use of front door, enter house from garage entrance”).

It may be noted that such recommendation of adding a sensor or a ramp to the dwelling 106 is only illustrative and should not be construed as a limitation. In other examples, the ambient health data platform 102a may also recommend and provide provision for, for example, integration of additional personal devices, dwelling modifications based on defined health and safety practices considered with predictive analysis of the occupant's health status, food products and/or health supplements for better lifestyle, addition of medications, addition of exercise or workout plan, personal preference items for the occupant 104.

In an example, the monitoring dataset is improved based on incorporation of the recommended enhancements by the occupant 104. For example, when the occupant 104 installs an additional device (such as, a sensor, a biosensor, or any other recommended device), or incorporates a dietary product (such as, a supplement, a food product, or any other item), or performs a recommended activity (such as, exercise, exit through the garage door, etc.), the ambient health data platform 102a may improve the monitoring dataset associated with the occupant 104. Alternatively, deterioration in health and/or dwelling condition may also be recorded to update the monitoring database of the occupant.

Embodiments of the present disclosure are related to a system for progressive environment and health evaluation and monitoring of the occupant 104. The system 102 performs monitoring of the environment within the dwelling 106 of the occupant 104. The system 102 performs the monitoring of the dwelling 106 using all available dwelling sensors, and monitoring of health and wellness of the occupant 104 via health/wearable sensors and home health devices. Using the data from all available dwelling/health sensors, the system 102 provides predictive recommendations to the occupant 104 and/or caregivers of the occupant 104 for preventive health and risk avoidance based upon past, present, and anticipated health risks.

In particular, the ambient health data platform 102a evaluates the historical and real-time environment data and/or health and wellness data in combination with historical data relating to the occupant 104 and the dwelling 106. The ambient health data platform 102a may analyze past/historic data relating to the occupant 104 for behavior or capability trends of the occupant 104. The ambient health data platform 102a amalgamates the environment data and the health and wellness data to provide current status of the occupant 104 and the dwelling 106. The ambient health data platform 102a may perform predictive analysis of health condition of the occupant 104, and provide proactive notifications to the occupant 104 and their support group, such as caregivers, nurse, and support community. The ambient health data platform 102a may register improvements or degradations in the condition of the dwelling 106 or the occupant 104, based on the behaviors and health status of the occupant 104 after incorporating the previous recommendations. To this end, the system 102 may consider ambient conditions and characteristics of the occupant 104 within their dwelling 106, based on health and wellness data relating to the occupant 104 and environment data relating to the dwelling 106, to generate recommendations for the occupant 104. Moreover, the system 102 may consider norms, standards of activity and behavior established for the specific occupant 104 in consideration of potentially exceptional data, such as disability data, allergy data, etc. to generate the recommendations. In an example, the system 102 may also consider boundary conditions established for the specific occupant 104 and their dwelling 106, personal and caregiver preferences or guidelines established for the specific occupant 104 and/04 the dwelling 106

In an example, the system 102 may be configured to provide predictive analysis of health condition of the occupant 104 to a social and support network of people who surround the occupant 104. For example, the support network may include data relating to family members, healthcare providers, case workers, religious or community contacts, designated guardians, physical therapist, occupational therapist, nurse, home-health providers, dietician, mental health providers, pharmacy, retail and delivery-based food providers, and house/personal assistants and other persons who may have a relevant relationship to the occupant 104. The system 102 may accept input from the occupant 104 and the support network to allow for customization and personalization of the recommendations for risk avoidance. The system 102 supports escalating and tiered notifications designed to ensure that communication of recommended enhancements is communicated and resolved and, if not resolved, escalating notifications to bring awareness to expanding members of the support network to enhance safety and risk avoidance for the occupant 104.

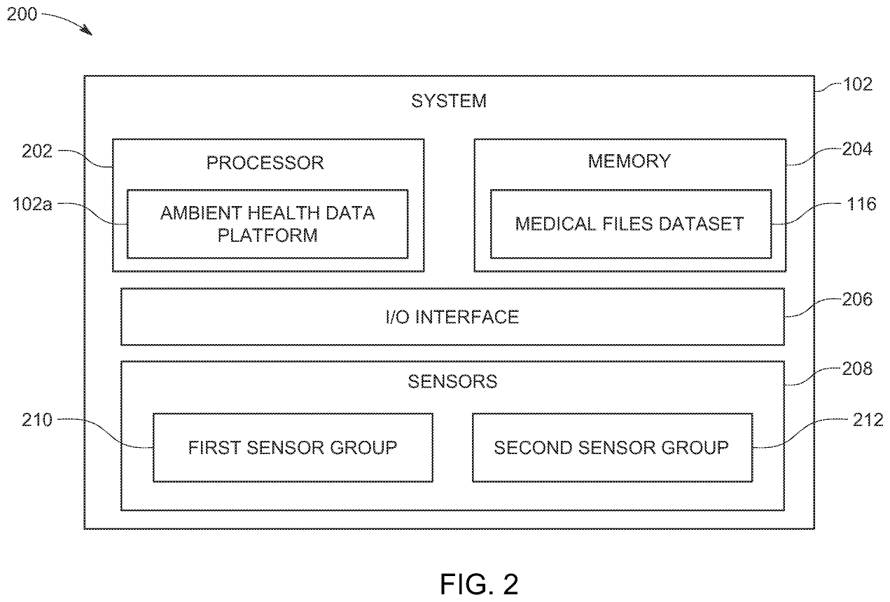

Referring to FIG. 2, there is shown a block diagram of the system 102, exemplarily illustrated in FIG. 1 for progressive evaluation and monitoring of environment and health of the occupant 104, in accordance with an example embodiment. FIG. 2 is explained in conjunction with FIG. 1.

The system 102 may include one or more processor 202 (referred to as a processor 202, hereinafter), a memory 204, an I/O interface 206, and a plurality of sensors 208 (referred to as sensors 208, hereinafter). The processor 202 may comprise the ambient health data platform 102a.

In accordance with an embodiment, the system 102 may store data that may be generated by the processor 202 while performing corresponding operation or may be retrieved from a database associated with the system 102, such as from the memory 204. In an example, the data may include sensor data, environment data, health and wellness data, monitoring dataset, occupant health and wellness evaluation, and enhancements.

For example, the sensors 208 may include a first sensor group 210 and a second sensor group 212. As described above, the first sensor group 210 collects environment data from the dwelling 106 of the occupant 104. The environment data includes the ambient and dwelling data collected by the plurality of dwelling sensors from the sensors 208 of the first sensor group 210. The plurality of dwelling sensors in the first sensor group 210 includes, but may not be limited to, temperature sensors, proximity sensors, cameras, active ultrasonic sensors, passive infrared sensors and other ambient sensors. The environment data collected from the plurality of dwelling sensors may include, for example, temperature data, motion detection data, water usage data, water leakage data, thermostat data, security data, surveillance data, electrical data, stove usage, refrigerator images, cooking, food preparation, refrigerator access, and the like. The environment data is received to establish the environmental norms for the occupant 104, and to provide information that supports proactive evaluation of the occupant's activities of daily living.

Further, the second sensor group 212 collects health and wellness data from the occupant 104. The health and wellness data includes the health condition data collected by the plurality of health sensors from the sensors 208 of the second sensor group 212. In some embodiments, the plurality of health sensors includes, for example, wearable devices such as smart watch, headphones, finger ring, health wrist band, fitness band, biosensor, and the like. In other embodiments, the plurality of health sensors corresponds to health/wellness monitoring devices such as a blood pressure cuff, oxygen sensor, weighing scale, glucose monitor, other standalone health statistic collection devices, data aggregation devices, smart phones, speakers, headsets and the like. The health and wellness data may include, but may not be limited to, respiratory rate, oxygen level, cardiac performance, heart rate, pulse rate, sleep patterns, glucose level, blood pressure, body temperature, and the like.

The memory 204 of the system 102 may be configured to store a dataset (such as, but not limited to, the medical files dataset 116, environment data, health and wellness data, occupant health and wellness evaluation, monitoring dataset, and the enhancements) associated with multiple occupants and corresponding dwellings. In accordance with an embodiment, the memory 204 may include processing instructions for processing environment data, health and wellness data, and medical files dataset. The medical files dataset may include past or historical medical records of the occupant 104 and/or past conditions of the dwellings 106.

In an example, the ambient health data platform 102a receives health and wellness data of the occupant 104 on a current and ongoing basis as well to capture changes in the health conditions of the occupant 104. Moreover, the ambient health data platform 102a receives at least one medical file from the medical files dataset 116. In an example, the medical file may include health records, clinical data and claims data that may be received from any clinical setting, care provider, or insurer that receives or authorizes treatment for the occupant 104. In addition, the medical files dataset 116 may be a centralized data center of any healthcare industry or a group of healthcare industries.

The ambient health data platform 102a receives environment data from the dwelling 106 via a plurality of dwelling sensors in the first sensor group 210, the health and wellness data of the occupant 104 through a plurality of health sensors in the second sensor group 212, and medical file relating to the occupant 104 from the medical files dataset. For example, the ambient health data platform 102a may also receive other information relating to the occupant 104, such as occupant data relating to the occupant 104 from the occupant 104 or the support network (for example, caregivers), and other historical data relating to the occupant 104 from health and environment data aggregation devices.

In one example, the ambient health data platform 102a receives certain data relating to the occupant 104 from a certified evaluator. For example, the certified evaluator may perform an assessment of the occupant 104, the dwelling 106 and the support network using standardized procedures and tools of the ambient health data platform 102a. In an example, the certified evaluator may be a person who has been trained and certified in the use of the evaluation tools included in the ambient health data platform 102a, or a medical professional. For example, the certified evaluator may provide information relating to evaluation of the occupant 104, the dwelling 106, and the support network of the occupant 104. Moreover, the evaluation of the occupant 104 may include, for example, consideration of physical and mental capabilities, mobility, strength, limitations, willingness to participate, ability to comprehend technologies, hearing, vision, ability to perform activities of daily living, and the like of the occupant 104. The evaluation of ambient characteristics of the dwelling 106 of the occupant 104 may include, for example, consideration of interior features, exterior features, trip/fall hazards, Steps, Railings, Doors, Windows, Water Sources, Halls, Walkways, Inclines/Declines, Obstacles, Lighting, Visible Differentiation of Surfaces, Cooking/Nutrition Appliances, Toilet, Bath/Shower, Media/Communication Devices, Smart Health Device, Smart Home Devices, and the like. The evaluation may also consider, such as a current caregiver, or support and social connections the occupant 104 to define the support network. The evaluation of the support network may include, for example, identification of Guardian(s) or additional caregivers, designated medical personnel, auxiliary care providers (such as physical therapist, occupation therapist, home health aide, etc.), friends and family, healthcare providers, case workers, religious or community contacts, dietician, mental health providers, pharmacist, retail and delivery-based food providers, handyman or house assistants, and the like.

Once updated data relating to the occupant 104 is received, the ambient health data platform 102a may be configured to perform evaluation of occupant data. The occupant data may include, but may not be limited to, the environment data, the health and wellness data, the medical file, data provided by the support network, data provided by the certified evaluator, and data provided by the occupant 104. In this regard, the ambient health data platform 102a may be configured to generate the monitoring dataset to organize the received data in a readable manner, or to improve evaluation or processing of the received data. Further, the ambient health data platform 102a may be configured to create an occupant health and wellness evaluation based on the monitoring dataset. The occupant health and wellness evaluation may include evaluation of the condition of the occupant 104 and the dwelling 106. For example, based on the occupant health and wellness evaluation, a chronic or acute disease occurring in the occupant 106, or food habits and lifestyle of the occupant 104 may be identified.

Thereafter, the ambient health data platform 102a may be configured to perform a predictive analysis to recommend enhancements to the monitoring dataset to improve the health and wellness evaluation of the occupant 104. For example, the predictive analysis may be performed on the occupant health and wellness evaluation to identify enhancements to improve condition of the occupant 104 and/or the dwelling 106. In an example, to improve the condition of the dwelling 106, certain repair work may be required. In another example, to improve health condition of the occupant 104 certain lifestyle changes and/or certain dietary changes may be required. In yet another example, to ensure comfort of the occupant 104, certain appliances or equipment, such as addition of ramp fixtures, ambient lighting, etc. may be required.

The ambient health data platform 102a may be configured to provide integrated e-commerce capabilities for the occupant 104 to order, fulfill, and integrate the recommended enhancements. In an example, the ambient health data platform 102a may be implemented on user equipment associated with the occupant 104 or the support network of the occupant 104. In this manner, the ambient health data platform 102a may provide provision for enabling the occupant 104 or the support network to order certain items, such as repair materials, food items, medicines, appliances, equipment, etc. for the occupant 104 through, for example, e-commerce websites. In this manner, the occupant 104 may fulfill the recommended enhancements and may further integrate the enhancements in their lifestyle for better health, wellness, comfort and lifestyle.

After the occupant 104 integrates or incorporates the recommended enhancements, the monitoring dataset may be improved. For example, the monitoring dataset may be improved to update the condition of the occupant 104 and/or the dwelling 106 after using the enhancements. For example, in case of an enhancement to be a medicine or a food supplement for a vitamin due to lack of such vitamin, the monitoring dataset may be improved to indicate that the occupant 104 may be no longer facing lack of such vitamin or condition of lack of such vitamin is addressed.

In an example, the processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally or alternatively, the processor 202 may include one or more processors configured in tandem via the bus to enable independent execution of instructions, pipelining and/or multithreading. Additionally or alternatively, the processor 202 may include one or more processors capable of processing large volumes of workloads and operations to provide support for big data analysis. In an example embodiment, the processor 202 may be in communication with the memory 204 via a bus for passing information among components of the system 102.

In an example, when the processor 202 is embodied as an executor of software instructions, the instructions may specifically configure the processor 202 to perform the algorithms and/or operations described herein when the instructions are executed. However, in some cases, the processor 202 may be a processor specific device (for example, a mobile terminal or a fixed computing device) configured to employ an embodiment of the present disclosure by further configuration of the processor 202 by instructions for performing the algorithms and/or operations described herein. The processor 202 may include, among other things, a clock, an arithmetic logic unit (ALU) and logic gates configured to support operation of the processor 202. The network environment, such as, 100 may be accessed using the communication interface 206 of the system 102. The communication interface 206 may provide an interface for accessing various features and data stored in the system 102.

Additionally, or alternatively, the processor 202 may include one or more processors capable of processing large volumes of workloads and operations to provide support for analysis and evaluation of environment data, health and wellness data, support network data, and certified evaluator data. In an example embodiment, the processor 202 may be in communication with the memory 204 via a bus for passing information among components coupled to the ambient health data platform 102a. In another embodiment, the processor 202 may also be connected via a networking mechanism to one or more virtual machine processors which are physically remote to enable cloud-based computing to assist with data processing, evaluation, application of artificial intelligence, rulesbased analysis, and the like. These virtual machines extend the local processing into the cloud seamlessly for additional compute power on-demand, or as scheduled.

The memory 204 may be non-transitory and may include, for example, one or more volatile and/or non-volatile memories. In other words, for example, the memory 204 may be an electronic storage device (for example, a computer readable storage medium) comprising gates configured to store data (for example, bits) that may be retrievable by a machine (for example, a computing device like the processor 202). The memory 204 may be configured to store information, data, content, applications, instructions, or the like, for enabling the system 102 to carry out various functions in accordance with an example embodiment of the present disclosure. For example, the memory 204 may be configured to buffer input data for processing by the processor 202. As exemplarily illustrated in FIG. 2, the memory 204 may be configured to store instructions for execution by the processor 202. As such, whether configured by hardware or software methods, or by a combination thereof, the processor 202 may represent an entity (for example, physically embodied in circuitry) capable of performing operations according to an embodiment of the present disclosure while configured accordingly. Thus, for example, when the processor 202 is embodied as an ASIC, FPGA or the like, the processor 202 may be specifically configured hardware for conducting the operations described herein.

In some example embodiments, the I/O interface 206 may communicate with the system 102 and displays input and/or output of the system 102. As such, the I/O interface 206 may include a display and, in some embodiments, may also include a keyboard, a mouse, a joystick, a touch screen, touch areas, soft keys, one or more microphones, a plurality of speakers, or other input/output mechanisms. In one embodiment, the system 102 may comprise user interface circuitry configured to control at least some functions of one or more I/O interface elements such as a display and, in some embodiments, a plurality of speakers, a ringer, one or more microphones and/or the like. The processor 202 and/or I/O interface 206 circuitry comprising the processor 202 may be configured to control one or more functions of one or more I/O interface 206 elements through computer program instructions (for example, software and/or firmware) stored on a memory 204 accessible to the processor 202. The processor 202 may further render notification associated with the recommended enhancements, such as reminders of medicines, tests to be performed, use of any appliance or equipment for ease or accessibility, personalized recommendations, etc., on a user equipment or audio or display associated with the occupant 104 via the I/O interface 206.

In some embodiments, the processor 202 may be configured to provide Internet-of-Things (IoT) related capabilities to the occupant 104 or other users of the system 102 disclosed herein. The IoT related capabilities may in turn be used to provide smart home solutions by providing real time warnings, big data analysis, and sensor-based data collection by using the cloud based system for providing accurate enhancements and ensuring safety, comfort, improved health and condition of the occupant 104 and the dwelling 106. The I/O interface 206 may provide an interface for accessing various features and data stored in the system 102.

In addition, the ambient health data platform 102a renders notifications or alerts to the support network based on predictive analysis and in case of emergent issues associated with the occupant 104. Also, the ambient health data platform 102a recommends mitigations and modifications that are specific to a risk profile developed room by room and for the dwelling 106 as a whole. Mitigations and modifications, i.e., enhancements that are recommended may be provisioned by the ambient health data platform 102a through integrated e-commerce. Time-to-completion for installation is decreased, and quality of configuration and integration of mitigations and modifications is increased through end-to-end evaluation. Further, the ambient health data platform 102a provides recommendations to the support network for improving health/wellness status of the occupant. As may be noted, the support network includes, for example, caregivers, contacts and/or family members, medical professional, etc. of the occupant 104. In addition, the support network corresponds to at least one of healthcare providers, case workers, religious or community contacts, designated guardians, physical therapist, occupational therapist, nurse, home-health providers, dietician, mental health providers, pharmacy, retail and delivery-based food providers, and house assistant or other person with relevant information regarding the occupant 104. The ambient health data platform 102a accepts input from the occupant 104 and the support network to tailor monitoring algorithms to allow for customization and personalization of the data combinations under analysis that result in monitoring health/wellness condition of the occupant 104 and providing predictive/preventative analytics for risk avoidance.

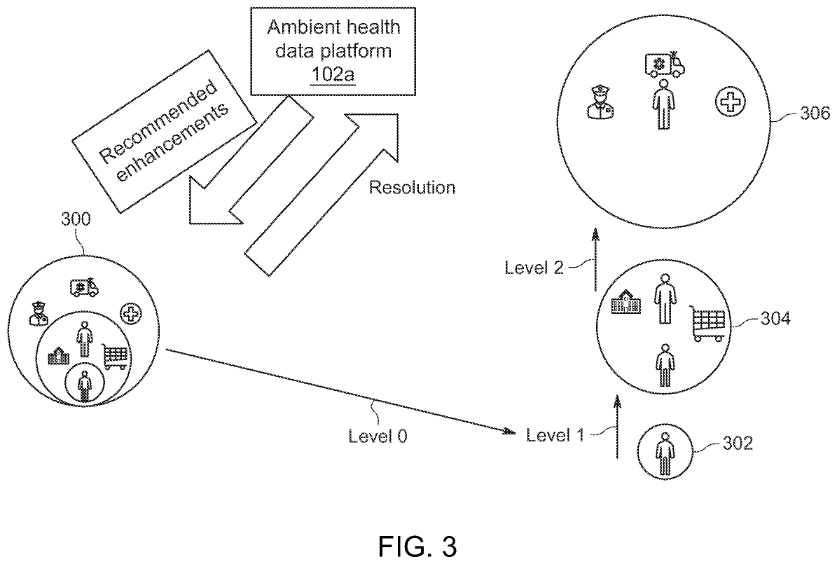

Based on the input received from the occupant 104 and the support network, the ambient health data platform 102a establishes an ambient health data learning environment. The ambient health data learning environment is established for each occupant/dwelling to define environment and health norms that are wholly specific and personalized to the data received from the occupant 104 and the dwelling 106, self-reported input, and support network data 208. The ambient health data learning environment maintains the historical set of data to provide an initial baseline of “norms” for the occupant/dwelling to perform periodic re-evaluation of known norms to assess trends/changes and provide consideration for re-base lining or notifications of deviation(s) outside of expected ratios. Retrospective analysis across different periods provides ability to further hone and refine occupant/dwelling specific configurations and produces new recommendations for dwelling 106 modifications, new ambient sensors, new personal devices, activity pattern changes, or mitigations and modifications to improve health/wellness and safety of the occupant 104. In addition, the ambient health data platform 102a renders notifications, or alerts to the support network based on customized levels. The customized levels of the support network are further explained in FIG. 3.

FIG. 3 illustrates a support network 300 associated with the occupant 104 and the ambient health data platform 102a, in accordance with an example embodiment. The support network 300 provides a flexible and responsive safety net for the occupant 104 that is fully customized to the competency of the occupant 104, urgency of the potential health/safety/wellness issues of the occupant 104 or the dwelling 106 and the resources available to the occupant 104. The support network 300 has customized and fully configurable notification types and escalation rules, which allows expanded support network that encompasses needs of the occupant 104 or the dwelling 106 for optimal health, safety and wellness. The ambient health data platform 102a provides recommended enhancements for improving condition of the dwelling 106 and/or the occupant 104, via notifications or alerts to the support network 300 based on the customized levels.

In an example, the customized levels in the support network includes level zero 302, level one 304, and level two 306. At level zero 302, the occupant 104 receives notifications or alerts, such as the notifications comprising recommended enhancements, from the ambient health data platform 102a. The ambient health data platform 102a provides direct communication to the occupant 104, or in case of a non-competent occupant, a designated “closest caregiver” may receive notifications and alerts to resolve non-emergency conditions, reminders, preventative, maintenance and warning issues. Once resolved, the occupant 104 or the closest caregiver may provide an intimation of the resolution of the non-emergency conditions, reminders, preventative, maintenance, or warning issues to the ambient health data platform 102a.

Further, if a recommended enhancement specified in notifications sent at level zero 302 are not resolved, then the recommended enhancements, the notifications or alerts are escalated to the level one 304 of the support network 300. At the level one 304, notifications are sent to additional caregivers, or designated medical personnel. In addition, notifications at level one 304 may also include system-to-system notifications in support of actions taken by or on behalf of the occupant 104, such as automated food orders, pharmacy orders, refills on supplies, and requests for assistance with installation of appliances or equipment, cleaning, cooking, etc. The occupant 104, the additional caregivers, or the designated medical personnel may initiate messages or requests for fulfilling the recommended enhancements and the ambient health data platform 102a may render notifications accordingly based on defined rules. Once resolved, the occupant 104, the additional caregivers, or the designated medical personnel may provide an intimation of the resolution of some or all of the recommended enhancements to the ambient health data platform 102a.

Continuing further, if a recommended enhancement or an issue specified in the notifications sent at level one 304 is not resolved or when boundary conditions are far exceeded, thereby indicating that health, safety, wellness of the occupant 104 and the dwelling 106 is in question, the notifications or alerts are escalated to level two 306 for requests for immediate help from any member in the support network 300. Subsequently, when resolved, the occupant 104, or any member in the support network 300 may provide an intimation of the resolution of some or all of the recommended enhancements or issues to the ambient health data platform 102a.

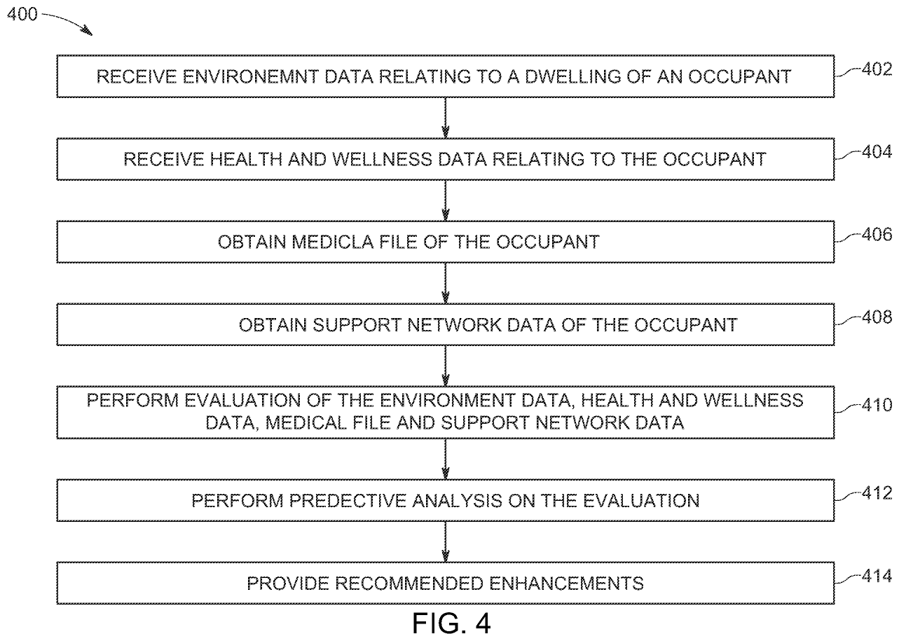

FIG. 4 illustrates a flow chart 400 of a method for evaluation and assessing of environment of the dwelling 106 and health of the occupant 104, in accordance with an example embodiment. In an example, the ambient health data platform 102a may be configured to assess, document, and evaluate the dwelling 106 and health and wellness of the occupant 104. Based on the evaluation, the ambient health data platform 102a may establish baseline features and risk profile for the occupant 104 and/or the dwelling 106, recommend enhancements or modifications for the dwelling 106 and/or the occupant 104, and enable purchase, installation, configuration, scheduling and testing of the enhancements.

The steps of the method 400 for evaluating and assessing the environment of the dwelling 106 and health of the occupant 104 may be performed using the components of the system 102.

At 402, environment data relating to the dwelling 106 is received. The dwelling 106 relates to the occupant 104. The environment data may include sensor data received from the first sensor group 112 or the plurality of dwelling sensors. The environment data may include, for example, temperature data, motion detection data, water usage data, water leakage data, thermostats data, security data, electrical data, stove usage, cooking, food preparation, refrigerator access, and the like. The plurality of dwelling sensors may include, for example, temperature sensors, proximity sensors, cameras, active ultrasonic sensors, passive infrared sensors and other ambient sensors. The environment data may be used to establish environmental norms and identify potential gaps, needs or areas of improvement that could be made to improve condition of the occupant 104.

In an example, the environment data may also be received from the occupant 104 and/or a certified evaluator. For example, the environment data may be received through the inputs and assessments of the certified evaluator.

At 404, health and wellness data relating to the occupant 104 is received. The health and wellness data may include sensor data received from the second sensor group 114 or the plurality of health sensors. For example, the plurality of health sensors may include one or more occupant wearable sensors (such as smartphones, smart watches, headphones, fitness tracker, health rings, ankle bands, health patch, hearing aid, etc.), or one or more home health devices (such as oximeter, standalone pulsometer, weighing scale, glucose meter, blood pressure monitoring device, etc.) The health and wellness data relating to the occupant 104 may include, but is not be limited to, respiratory rate, oxygen level, cardiac performance, heart rate, pulse rate, sleep patterns, glucose level, blood pressure, and body temperature.

In an example, the health and wellness data relating to the occupant 104 may also be received from the occupant 104 and/or a certified evaluator. For example, the health and wellness data of the occupant 104 may also include historical data from health or dwelling data aggregation devices. The historical data may include, for example, health statistics, informatics, caloric intake measurements, activity/exercise levels from self-reported data, or other data collected by the aggregation devices.

At 406, medical file of the occupant is obtained. For example, past medical records of the occupant 104 may be accessed to obtain the medical file of the occupant form the medical files dataset 116. The medical files dataset may be maintained by, for example, hospital, clinician, clinical setting, laboratory, health service provider, insurer, a professional from which the occupant 104 is receiving treatment or has authorized treatment or payment against any treatment. For example, the medical files dataset 116 may be maintained by a centralized data center of any healthcare industry or a group of healthcare industries, healthcare data sharing organizations or regional health information networks.

At 408, support network data is obtained relating to the support network 300 of the occupant 104. The support network data may include, for example, identification and contact information of guardian(s), additional caregivers, designated medical personnel, auxiliary care providers (physical therapy, occupational therapy, home health aide), friends, family members, healthcare providers, case workers, religious or community contacts, dietician, mental health providers, pharmacist, retail and delivery-based food providers, handy-man or house assistants or other persons with a relevant relationship to the occupant 104. In an example, contact information and participation requirements for each member of the support network 300 may be predefined for identifying the members of the support network for the occupant 104. Moreover, contact information of the members of the support network 300 may be registered in customized levels, as described in FIG. 3. Based on the customized levels and a nature of a notification or alert or a recommendation, one, few or all members of the support network 300 may be notified regarding the health status of the occupant 104.

At 410, evaluation of the environment data, the health and wellness data, medical file and support network data is performed. In an example, baseline status of occupant health and wellness may be established based on the evaluation. In an example, the environment data, the health and wellness data, the medical file and the support network data may be evaluated to create occupant health and wellness evaluation using one or more artificial intelligence-based algorithms. In another example, the environment data, the health and wellness data, the medical file and the support network data may be evaluated to create occupant health and wellness evaluation by a certified evaluator.

In an example, the ambient health data platform 102a performs multi-variate analysis of dwelling 103 and occupant 107 characteristics, i.e., environment data, the health and wellness data, medical file and support network data. In some embodiments, this analysis will include the use of artificial intelligence, rules assessment, comparisons to best practices, boundary condition analysis, using analytic methods that include: qualitative, quantitative, content, contextual, regressive, mathematical, statistical, time series, decision tree, discriminant, fuzzy logic, neural, factor, dispersion, evolutionary and the like. The data inputs being analyzed will include any data that may be provided via the evaluator discussion and observation, data provided by the occupant 104 or support network 300, data received from the sensors 110 (such as the plurality of dwelling sensors and the plurality of health sensors). The analysis may consider the existence of certain devices and usage methods and ways in which those devices are being employed by the occupant 104 and the dwelling 106, absence of certain devices, the absence of data types and variables that will also be considered with scenario considerations, what-if analysis, best-case analysis or worst-case analysis that can compare potential outcomes possible if additional/better data were available.

At 412, predictive analysis is performed on the evaluation. In an example, assessments and results from the predictive analysis of health condition of the occupant 104 and current state of the dwelling 106 may be recorded. The predictive analysis may be performed to identify shortcomings in the health of the occupant 104 and/or conditions of the dwelling 106, and further identify ways to correct such shortcomings in effective manner.

In an example, the predictive analysis of the health conditions, capabilities and limitations of the occupant 104 and current state of the dwelling 106 may be performed to identify modifications and enhancements for augmentation in the dwelling 106 or for the occupant 104. For example, ways to correct the shortcomings in the health of the occupant 104 and/or the condition of the dwelling 106 may be identified as recommended enhancements for the occupant 104.

In an example, the predictive analysis may be performed using analytical methods. The analytical methods include, for example, qualitative, quantitative, content, contextual, regressive, mathematical, statistical, time series, decision tree, discriminant, fuzzy logic, neural, factor, dispersion, evolutionary and the like.

In an embodiment, providing the predictive analysis at a time of initial evaluation and assessment of the occupant 104 and the dwelling 106 allows the ambient health data platform 102a to bundle recommended modifications in logical ways that may facilitate implementation and potentially save time and money. For example, if the dwelling 106 has entry steps that are not currently a problem for the occupant 104, but the ambient health data platform 102a considers that the occupant 104 currently has low-grade mobility issues, a future enhancements recommendation may include door way and entry way modifications to facilitate entry (such as door replacement to provide a window for better visibility/lighting, door opener, door widening, motion sensor, elimination of steps, etc.). If multiple modifications to the entryway are potentially valid for this dwelling 106, the ambient health data platform 102a may aggregate the enhancements in a sequence that reduces effort overall so that, for example, if door replacement is recommended so that a window is added, the doorway widening is also recommended at the same time so that the entry is better configured to accommodate a future door opener or ramp.

At 414, recommended enhancements may be provided. The recommended enhancements may be provided with provisioning of direct purchase and fulfillment. For example, the recommended enhancement may include provisioning of an additional sensors to be installed in the dwelling 106, additional personal device to be connected for monitoring or treating the occupant, dwelling modifications based on defined health and safety practices considered with occupant's health status, dietary restrictions, and devices based on personal preferences, and the like. The recommended enhancements may be provided for modifications, improvements, or additions to the dwelling 106 and/or for behavior tracking and pattern or activity changes for the occupant 107. For example, based on best practices for health and house, standards of care, behavioral recommendations, occupant preferences and support network data with customized levels to create personalized boundaries, limit conditions, alert conditions and escalation rules, the recommended enhancements may be provided to the occupant 104 and/or the support network 300.

In an example, the recommended enhancements may be provided in the form of a customized report that reflects the totality of the amalgamated analysis of the occupant 104 and their dwelling 106. For example, if the occupant 104 has a limiting condition (such as “reduced mobility”) and their dwelling 106 currently has stairs that must be used to enter the front door, but the garage entry has only 1 step to enter the house, the recommendations may include both “install ramp to front door” and “avoid use of front door, enter house from garage entrance”.)

In an example, consideration of the occupant 104 and the dwelling 106 may occur singularly to create entity-specific recommendations. For example, an occupant with balance issues may be recommended a personal device that may detect a fall. In another example, a dwelling that lacks working smoke detectors may be recommended smoke/heat sensors. These entity-specific risks are recorded by the ambient health data platform 102a. In an example, consideration of the occupant 104 and the dwelling 106 may be amalgamated to perform analysis to assess the ergonomics, risk factors, health/welfare/safety factors in relationship to the interaction of the occupant 104 and their ambient environment. The amalgamated factors may allow for highly specific and personalized outcomes and recommendations that may be time-phased and risk stratified. For example, immediate and high impact recommendations for items which should be considered because they will improve health/welfare/safety of the occupant 104 and immediately lower the risk of injury. Further, other categories of recommendations may include predictive and preventative outcomes that the ambient health data platform 102a considers to be “highly likely” or “likely” to be applicable in the future for the occupant 104 and/or the dwelling 106 combination. These amalgamated occupant 104 and/or dwelling 106 risks may also be recorded by the ambient health data platform 102a.

In an example, the certified evaluator, the occupant 104 and/or members of the support network 300 may select one or more desired enhancement from the report recommendations. In a case where the selected enhancement relates to purchase of a product or a service, the occupant 104 and/or the members of the support network 300 may be provided with direct links to purchase the recommended product. The occupant 104 and/or the members of the support network 300 may also be provided with provision of scheduling professional support, sending requests to third-party support entities (for example, access to food delivery and/or grocery delivery apps, account support for automated grocery delivery, etc.).

In an example, the customized report includes the results of the analysis performed by the ambient health data platform 102a. The customized report may also include potential dwelling risk mitigations such as, but not be limited to, immediate mitigations enhancements (for example, remove trip hazards, change doors or locks, change occupant pattern of sleep, etc.), non-construction enhancements (for example, signs, reflective tape, etc.), construction enhancements (for example, ramp, grab-bar, steps modification, etc.), sensors enhancement and integration or enhancement of existing capabilities of sensors or devices, and the like. Moreover, enhancements associated with the support network 300 may also be recommended, such enhancements may include, but not be limited to, physical therapy, occupational therapy, language therapy, third-party service enrollment (grocery/RX delivery, rideshare, etc.), in-home assistance (such as for cooking, cleaning, home health aide, etc.), behavioral health/therapy, and the like. In certain cases, potential occupant enhancements may be recommended, such as personal device recommendations, self-directed strength or balance or fitness programs, assistive mobility devices, self-directed health/nutrition, other provider-led or assisted programs, and the like.

As the occupant 104 or members of the support network 300 selects enhancements to be fulfilled or which has been fulfilled, the report may dynamically adjust recommendations so that dependent or duplicative choices are adjusted. For example, if multiple choices exist to modify a doorway, and one is selected, then the other choices will cease to be available. As recommendations are completed, changes are noted in the monitoring dataset of the occupant 104 and the dwelling 106. The monitoring dataset may be adjusted based on enhancement monitoring rules, boundary conditions, notification sets and escalation rules established for the occupant 104 and/or the dwelling 106.

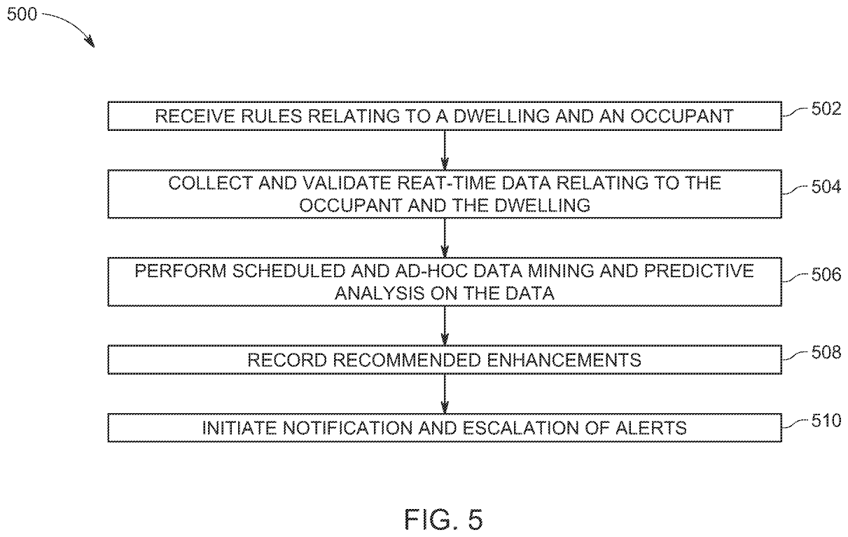

FIG. 5 illustrates a flow diagram 500 of a method for monitoring the dwelling 106 and health and wellness status of the occupant 104, in accordance with an example embodiment. The method for monitoring environment of the dwelling 106 and the occupant 104 is performed using the components of the system 102.

In an example, the occupant 104 may register on the ambient health data platform 102a to improve safety and health in their dwelling 106. After registering, an evaluation team may be created by the ambient health data platform 102a for the occupant 104. The evaluation set may include, for example, one or more evaluators, one or more evaluation test forms, one or more evaluation requests (such as evaluations to be fulfilled by a third-party, for example, a lab, a medical professional, a diagnostic center, etc.). For example, the evaluation set may also include additional evaluators due to special needs, health considerations, safety considerations, physical or mental limitations of the occupant 104, and so forth. For example, data received from the evaluation set may be used to determine initial baseline features of the occupant 104 and/or the dwelling 106.

In an example, the evaluators may perform the initial evaluation of the occupant 104 around the basic capabilities of the occupant 104 and the occupant's health, and the dwelling 106 of the occupant 104. For example, the evaluation may be performed using a set of predefined assessment tools. The set of predefined assessment tools may include systematic analyses that use Lean principles and processes to provide standard instructions, standardized repeatable steps, and to ensure that all assessment steps are completed for every assessment, across all types of assessments in all locations.

In an example, the set of predefined assessment tools may include occupant assessment tools to assess capability of the occupant 104. Such occupant assessment tools may include, for example, observable, collectable and measurable data and evidence related to the ability of the occupant 104 to interact with their environment, use device(s) or equipment available at their disposal to ease their daily life, be self-sufficient in meeting their own basic living needs, provide in or participate in their own healthcare. In some embodiments, the occupant assessment tools may assess the occupant 104 based on demonstrations of capabilities of the occupant 104 to perform household duties (such as cooking, picking up items off the floor, etc.) or physical activities (such as, climbing stairs, opening doors, getting up from sitting position, etc.). In other embodiments, a member of the support network 300 may be involved for assessing the capabilities of the occupant 104 to provide feedback and information for the occupant assessment tools when the occupant 104 may be incapable of communicating or demonstrating.

In another example, the set of predefined assessment tools may include dwelling assessment tools to assess capability of the dwelling 106. The dwelling assessment tools may be used for evaluating the dwelling 106. The dwelling assessment tools allow for the capture of observable, collectable and measurable data and evidence related to interior and exterior features of the dwelling 106 or residence of the occupant 104 and surrounding space, including roads, traffic, cameras, neighboring buildings, shops, etc. The dwelling assessment tools may also be used to capture pictures and measurements, annotate with drawings and notes, and identify existing and potential safety risks based on industry best practices, known safety standards, human factors issues, reported issues, and occupant capabilities.

In an example, the evaluator or the ambient health data platform 102a may use the set of predefined assessment tools to capture initial evaluation of the occupant 104 and/or the dwelling 106. Visual identification and narrative descriptions of environmental, ambient dwelling features and occupant features may be recorded on the ambient health data platform 102a.

In an example, results of the evaluation of the occupant 104 and the dwelling 106 are ingested into the ambient health data platform 102a to consider the interactions, touch points, known and established risks based on the skills and capabilities of the occupant 104 and the ambient characteristics of the dwelling 106. The ambient health data platform 102a may use defined system rules, guidelines, best practices for home safety, senior/disabled persons' safety guidelines, health/wellness guidelines, human factors guidelines, inputs from the occupant 104 and the support network 300 to perform predictive analysis.

In an example, the evaluator may determine the availability of aggregation device(s) and medical files relating to the occupant 104. The evaluator may assist the occupant 104 and/or members of support network 300 to enable the ambient health data platform 102a to gain access or identify integration options for such data sources. In some instances, the host or devices used for data aggregation may be common, known and have accessible or published application programming interfaces (APIs) which can be activated within the ambient health data platform 102a. The evaluator may also evaluate the current telecommunications infrastructure, internet connectivity, data transmission speeds, Wi-Fi speed and coverage areas within the dwelling 106 to ensure the sensors 110 within the dwelling 106 and/or associated with the occupant 104 is able to transmit data in real-time.

At 502, receive rules for the occupant 104 and the dwelling 106. The rules may include, for example, a set of monitoring rules, boundary conditions, notification sets and escalation rules for the occupant 104 and the dwelling 106. In an example, validity of the received rules may be compared against the last known conditions of the occupant 104 and the dwelling 106. Once validated, the conditions provided by the occupant 104 may become the rules set against which the last known conditions. Further, any future incoming data may be evaluated against or validated against the presently set conditions to set new rules.

Further, the ambient health data platform 102a may provide configurable room and space definitions that enable rapid and repeatable capture of environmental and structural features associated with the dwelling 106 by the plurality of dwelling sensors in the first sensor group 112, and health and wellness data of the occupant 104 by the plurality of health sensors in the second sensor group 114.

For example, the ambient health data platform 102a may use the result of the initial evaluation of the occupant 104 and/or the dwelling 106 to determine the rules for the occupant. The initial evaluation may be generated based on, for example, result of the set of predefined assessment tools, any other assessment by the evaluators or the evaluation set or the support network 300, etc. Based on the initial evaluation, the ambient health data platform 102a may learn and retain the baseline characteristics of the occupant 104 and the dwelling 106.

In certain cases, such as in case of any error, anomalies, questions, or risks in results of the initial evaluation, and/or setting of incorrect rules based on the initial evaluation of capabilities the occupant 104, etc., the ambient health data platform 102a may provide feedback to the evaluator, the occupant 104, or the support network 300. In certain cases, the ambient health data platform 102a may prompt the evaluator to gather additional information from further occupant 107 or dwelling 103 if the ambient health data platform 102a predicts that a future modification may be appropriate.

At 504, collect and validate real-time data. For example, the data may include most recent, real-time or incoming health and wellness data, environment data, medical files, and support network data obtained from the sensors 110, the support network 300, the occupant 104, and an evaluator. In an example, monitoring dataset may be generated based on the validated real-time data relating to the occupant 104 and/or the dwelling 106.

In an example, the evaluator may identify, test, and validate the use of the sensors 110 (comprising the plurality of dwelling sensors and the plurality of health sensors) to initiate integration of health and wellness data and dwelling data for monitoring health and wellness of the occupant 104. For example, use and location of the plurality of dwelling sensors and the plurality of health sensors in the dwelling 106 and/or on the occupant 104 may be noted. In certain cases, the evaluator may also determine inventory, identify, test and validate the certain personal devices and other equipment currently in use by the occupant 104. Existing personal devices, sensors, potential additional personal devices, potential sensors and other equipment that are not fully being utilized may also be noted for final recommendation or receiving real-time data relating to the occupant 104 and/or the dwelling 106. Based on the identified sensors 110, data may be collected in real time to monitor and evaluate conditions of the dwelling 106 and the occupant 104.

At 506, perform scheduled and ad-hoc data mining and predictive analysis on the collected and validated data. For example, the predictive analysis may include comparing profile data of the occupant 104 and/or the dwelling 106 with the incoming data streams or updated monitoring dataset. For example, based on the rules set by the occupant 104 and/or the members of the support network 300 and other historical data, profile data may be set-up for the occupant 104 and the dwelling 106. Further, the data may be received from the sensors 110 within the dwelling 106 and/or associated with the occupant 104, medical files dataset 116, health-dwelling data aggregation device(s), support network, self-reported data. The predictive analysis may consider a current state of the occupant 104 and/or the dwelling 106, based on the received data. Further, the current state may be compared with prior state to predict future state of the health, wellness and safety of the occupant 104 in consideration and reflection of the status of the dwelling 106.

The analytical method for performing the predictive analysis may include qualitative, quantitative, content, contextual, regressive, mathematical, statistical, time series, decision tree, discriminant, fuzzy logic, neural, factor, dispersion, evolutionary and the like. These methods are used to assess rates of change, boundary condition violations, anomalous behaviors, pattern deviations, requirements, and the like to detect situations that indicate risk to safety/wellness

At 508, recommended enhancement of the predictive analysis is recorded in real-time. The enhancement may be used as basis for any recommendations, notifications and escalations, and the enhancement are retrievable at any point in the future.

At 510, notification and escalation of alerts is initiated. For example, the notification and alert may be provided to the occupant 104 and/or member of the support network 300. For example, notifications may be rendered to the support network based on the enhancement identified in the predictive analysis, emergent situations, rules set for the occupant 104, customization levels, and a nature the enhancement.

In an example, the enhancement may include recommendations for improving occupant's health, wellness and safety status. Recommendations include but may not be limited to “replace the kitchen tap as it has leakage”, “check temperature sensor as it is showing a constant temperature from past 5 days”, and “go sleep at 9 pm today, as your sleeping pattern is disturbed”. In some embodiments, notifications include “cardiac health declining, assist the occupant”, “occupant has fallen in the kitchen, assist immediately” or “check occupant's nutrition status due to glucose reading, depressed overall activity and lack of activity in the kitchen” and the like.

For example, the actual and the predicted health, wellness and safety factors are communicated based on the independent risk factors as well as the combined or amalgamated risk factors indicated by the predictive analysis and data mining of the received data. Each factor defined in the alerts or notifications allows for consideration of the different health and well-being parameters independently and as a whole so that significant deviations from expected norms will cause escalation, and a combined impact of potentially minor deviations are more significant when considered together. In an example, minor deviations in sleep patterns or movement within the dwelling 106 may not be significant when considered singularly; however, when combined with reduced cardiac strength or Oxygen capacity and uncharacteristic or unexplained environmental changes such as erratic thermostat changes or night-time entrance/exit data from the dwelling 106 may indicate confusion or impacted mental state.

In an example, the communication of notifications to the support network may occur and which may initiate, for example, improvement or enhancement or rescue for the occupant operations for the occupant 104.

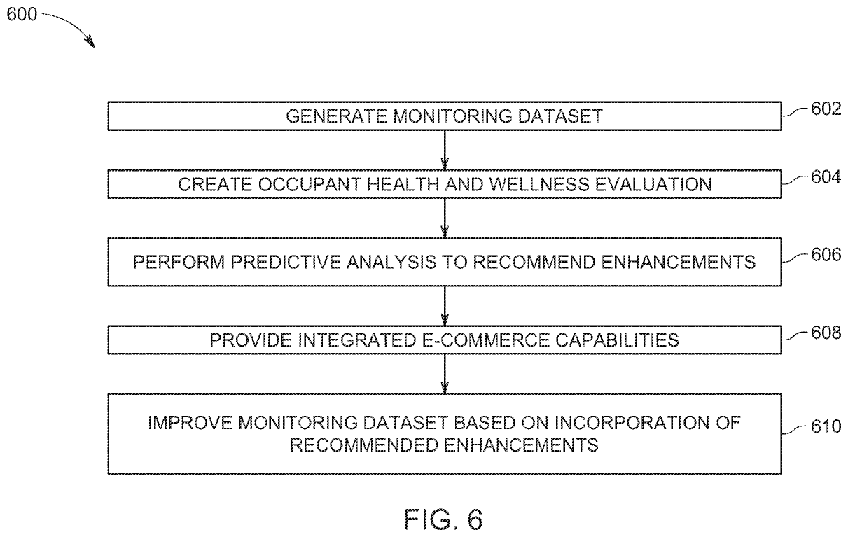

FIG. 6 illustrates a method 600 for progressive evaluation and monitoring of environment and health of the occupant 104, in accordance with an example embodiment.

At 602, a monitoring dataset is generated. In this regard, data is received the first sensor group 112, the second sensor group 114, and the medical files dataset 116. The first sensor group 112 collects environment data relating to the occupant 104. The first sensor group 112 comprises a plurality of dwelling sensors. The second sensor group 114 collects health and wellness data relating to the occupant 104. The second sensor group 114 comprises a plurality of health sensors. The medical files dataset 116 comprises at least one medical file relating to the occupant 104. For example, the monitoring data set may include real-time data relating to the occupant 104 and the dwelling 106.

At 604, an occupant health and wellness evaluation is created based on the monitoring dataset. The occupant health and wellness evaluation may include evaluation of the received data against rules defined for the occupant 104 and/or the dwelling. For example, the occupant health and wellness evaluation may indicate shortcomings in conditions of the occupant 104 and/or dwelling 106.

At 606, predictive analysis is performed to recommend enhancements to the monitoring dataset to improve the health and wellness evaluation of the occupant 104. For example, the enhancements may be identified to improve health, well-being and safety of the occupant 104 and/or condition of the dwelling 106.

At 608, integrated e-commerce capabilities are provided for the occupant 104 to order, fulfill, and integrate the recommended enhancements. For example, the integrated e-commerce capabilities may be provided to purchase a product (such as additional sensor, additional equipment, etc.) or a service (such as service appointment, medical appointment, etc.) that may be recommended for the occupant 104. For example, the enhancements may be provided to the support network 300 based on customization level to ensure safety and wellness of the occupant 104.

At 610, the monitoring dataset is improved based on incorporation of the recommended enhancements. The enhancements help occupants in reducing future risks to their health that are predicted, tackle current health conditions, as well as maintain good health. Additionally, the disclosed system and technique allow for the alerting of the occupant's support network 300. These alerts include predicting improvements in condition of the occupant 104 or the dwelling 106 with varying levels of necessity, such as alerting the resident's nearest caregiver to adjust their meal plan.

In some cases, the disclosed system and technique provide improvements for the occupant's 104 dwelling 106, for example, configure new temperature sensor in hall or change the dwelling entry from main gate to garage gate. This is useful if the occupant continues to walk properly. Even in the case that an occupant's routine is enhanced, for example, configure new home device may prevent the poor sleep cycle, unsuitable food consumption, inappropriate workout and exercises. Furthermore, the monitoring dataset is used to provide improvements to the occupant, dwelling, or both, and may prevent the predicted health hazards in the future.

Accordingly, blocks of the flowcharts 400, 500 and 600 support combinations of means for performing the specified functions and combinations of operations for performing the specified functions. It will also be understood that one or more blocks of the flowcharts 400, 500 and 600, and combinations of blocks in the flowcharts 400, 500 and 600, can be implemented by special purpose hardware-based computer systems which perform the specified functions, or combinations of special purpose hardware and computer instructions.

Alternatively, the system 102 may comprise means for performing each of the operations described above. In this regard, according to an example embodiment, examples of means for performing operations may comprise, for example, the processor 202 and/or a device or circuit for executing instructions or executing an algorithm for processing information as described above.

On implementing the methods 400, 500 and 600 disclosed herein, the end result generated by the system 102 is a tangible evaluation and monitoring of the conditions of the occupant 104 and the dwelling 106. The evaluation and monitoring of the ambient conditions of the dwelling 106 and condition and characteristics of the occupant 104 is crucial to provide accurate and complete information relating to occupant to ensure health, safety and well-being of the occupant 104 based on condition of the occupant as well as condition of the dwelling 106, thereby ensuring occupant safety.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A system for progressive evaluation and monitoring of environment and health of an occupant, the system comprising:
a first sensor group to collect environment data relating to the occupant, the first sensor group comprising a plurality of dwelling sensors;
a second sensor group to collect health and wellness data relating to the occupant, the second sensor group comprising a plurality of health sensors;
a medical files dataset comprising at least one medical file relating to the occupant; and
an ambient health data platform configured to:
generate a monitoring dataset based on the first sensor group, the second sensor group, and the medical files dataset;
create an occupant health and wellness evaluation based on the monitoring dataset;
perform a predictive analysis to recommend enhancements to the monitoring dataset to improve the health and wellness evaluation of the occupant; and
provide integrated e-commerce capabilities for the occupant to order, fulfill, and integrate the recommended enhancements,
wherein the monitoring dataset is improved based on incorporation of the recommended enhancements by the occupant.

2. The system of claim 1, wherein the plurality of dwelling sensors comprises at least a camera.

3. The system of claim 1, wherein the plurality of health sensors comprises at least one of: one or more occupant wearable sensors or one or more home health devices.

4. The system of claim 1, wherein the at least one medical file comprises at least one of: personal health records, electronic medical record, medical history, or third-party data obtained from an occupant caregiver relating to the occupant.

5. The system of claim 4, wherein the third-party data obtained from the occupant caregiver includes data from at least one of: family members or medical professionals.

6. A system for progressive evaluation and monitoring of environment and health of an occupant, the system comprising:
a first sensor group to collect environment data relating to the occupant, the first sensor group comprising a plurality of dwelling sensors;
a second sensor group to collect health and wellness data relating to the occupant, the second sensor group comprising a plurality of health sensors including occupant wearable sensors and home health devices;
a medical files dataset comprising at least one of: personal health records, electronic medical record, available medical history, and third-party data obtained from an occupant caregiver relating to the occupant; and
an ambient health data platform configured to:
generate a monitoring dataset based on the first sensor group, the second sensor group, and the medical files dataset;
create an occupant health and wellness evaluation based on the monitoring dataset;
perform a predictive analysis to recommend enhancements to the monitoring dataset to improve the health and wellness evaluation of the occupant; and
provide integrated e-commerce capabilities for the occupant to order, fulfill, and integrate the recommended enhancements,
wherein the monitoring dataset is improved based on incorporation of the recommended enhancements by the occupant.

7. The system of claim 6, wherein the plurality of dwelling sensors comprises at least a camera.

8. The system of claim 6, wherein the third-party data obtained from the occupant caregiver includes data from at least one of: family members or medical professionals.

9. A method for progressive evaluation and monitoring of environment and health of an occupant, the method comprising:
generating, using an ambient health data platform, a monitoring dataset based on a first sensor group, a second sensor group, and a medical files dataset, wherein
the first sensor group collects environment data relating to the occupant, the first sensor group comprising a plurality of dwelling sensors,
the second sensor group collects health and wellness data relating to the occupant, the second sensor group comprising a plurality of health sensors, and
the medical files dataset comprise at least one medical file relating to the occupant;
creating, using the ambient health data platform, an occupant health and wellness evaluation based on the monitoring dataset;
performing, using the ambient health data platform, a predictive analysis to recommend enhancements to the monitoring dataset to improve the health and wellness evaluation of the occupant;
providing, using the ambient health data platform, integrated e-commerce capabilities for the occupant to order, fulfill, and integrate the recommended enhancements; and
improving, using the ambient health data platform, the monitoring dataset based on incorporation of the recommended enhancements.

10. The method of claim 9, wherein the occupant health and wellness evaluation is performed by an evaluator.

11. The method of claim 9, wherein the plurality of health sensors comprises at least one of: one or more occupant wearable sensors or one or more home health devices.

12. The method of claim 9, wherein the at least one medical file comprises at least one of: personal health records, electronic medical record, medical history, or third-party data obtained from an occupant caregiver relating to the occupant.

13. The method of claim 12, wherein the third-party data obtained from the occupant caregiver includes data from at least one of: family members or medical professionals.

14. The method of claim 9, the method further comprising:
performing, using the ambient health data platform, an occupant health and wellness re-evaluation based on the improved monitoring dataset after the incorporation of the recommended enhancements.

15. The method of claim 9, wherein the recommended enhancements comprise an additional health sensor for environment and health monitoring of the occupant.

16. The method of claim 9, wherein the monitoring dataset comprises historical data obtained from the plurality of dwelling sensors, the plurality of health sensors, and the medical files dataset.

17. The method of claim 9, wherein the ambient health data platform receives data from the first sensor group and the second sensor group via a communication network.

18. The method of claim 9, wherein the occupant health and wellness evaluation includes an assessment of physical capabilities of the occupant.

19. The method of claim 9, wherein the occupant health and wellness evaluation includes an assessment of the plurality of dwelling sensor, the plurality of health sensors, and the medical files dataset of the occupant.
